(19) United States
(12) Patent Application Publication
(10) Pub. No.: US 2025/0009181 A1
(43) Pub. Date: Jan. 9, 2025

(54) METHOD FOR DETERMINING A STATE PARAMETER, METHOD FOR DETERMINING A QUANTITY, DETECTION DEVICE AND FRYER

(71) Applicant: Testo SE & Co. KGaA, Titisee-Neustadt (DE)

(72) Inventors: Martin Zubler, Lenzkirch (DE); Benjamin Landenberger, Freiburg (DE)

(73) Assignee: Testo SE & Co. KGaA, Titisee-Neustadt (DE)

(21) Appl. No.: 18/751,720

(22) Filed: Jun. 24, 2024

(30) Foreign Application Priority Data
Jul. 5, 2023 (DE) 102023117755.9

(51) Int. Cl. A47J 37/12; G01N 33/03

(52) U.S. Cl. CPC A47J 37/1266; G01N 33/03

(57) ABSTRACT

A method for determining a state parameter of a frying medium (3), in which at least one event parameter which describes at least one frying event (10) is measured and processed for the determination, as well as a method for determining the quantity of water entrained into a frying medium (3) during a frying process and/or frying event (10) in which a foam formation is measured and evaluated during the frying process and/or frying event 10 are provided. A detection device (11) for detecting frying events (10) of a fryer (12) having a sensor (13) fitted above the fryer (12) and/or directed toward the fryer (12) and having a computer, in which the detection device (11) is configured to carry out the method is also provided. A fryer (12) which has a detection device (11) is further provided.

### CROSS-REFERENCE TO RELATED APPLICATIONS

This application claims priority to German Patent Application No. DE 110 2023 117 755.9, filed Jul. 5, 2023, which is incorporated herein by reference as if fully set forth.

### TECHNICAL FIELD

The invention relates to a method for determining a state parameter of a frying medium.

The invention further relates to a method for determining the quantity of water entrained into a frying medium during a frying process.

The invention further relates to a detection device for detecting frying events of a fryer.

The invention further relates to a fryer.

### BACKGROUND

In order to fry a frying food item, said frying food item is placed in a frying medium that is normally usable multiple times. This normally involves vegetable fats and/or animal fats and/or fatty oils. Depending on the composition, the frying medium contains saturated and/or unsaturated fatty acids, normally in the form of triacylglycerols. During a frying process, the frying medium is subjected to thermal and/or chemical loads which adversely affect the quality thereof as the frying process progresses. The state of the frying medium is regularly checked for quality assurance and for compliance with legal provisions. Countermeasures, for example replacement of the frying medium, can thus be instigated if the frying medium is in an inadequate state. This check can be carried out, on one hand, through subjective evaluation by persons, and, on the other hand, through objective measurement of quality-related parameters. Quality-related parameters can be determined, for example, by means of measuring strips or test strips, wherein this type of determination is comparatively expensive and is not resource-efficient, and, due to a subjective reading of the strips, is inaccurate. The quality-related parameters can be, for example, the mass fraction of polar materials (% TPM) or the mass fraction of free fatty acids (% FFA). In practice, the measurement of these parameters represents a time-consuming and costly process, which furthermore requires suitable personnel.

### SUMMARY

The object of the invention is to simplify the determination of quality-related parameters of frying media.

One or more of the features disclosed herein are provided according to the invention in order to achieve the aforementioned object. In particular, in order to achieve the aforementioned object in methods of the type described above, it is therefore provided according to the invention that at least one event parameter which describes at least one frying event is measured and processed in order to determine a state parameter of a frying medium.

The event parameter is state-independent here. This means that the event parameter can be the same for frying events of the same type, even though the state of the frying medium differs in the individual frying events. A frying process can consist of a plurality of frying events, wherein a frying event continues from the addition of a frying food item to a frying medium until the removal of the frying food item. The event parameter can describe an effect of the frying event on the frying medium. A state parameter of a frying medium can thus be inferred from an event parameter. It is therefore not necessary to directly capture, for example measure, the state parameter that is to be determined. A load and/or a regeneration of the frying medium can thus be determined and/or monitored by means of the state parameter.

In one advantageous embodiment, it can be provided that measured event parameters are accumulated.

Event parameters can, for example, be counted and/or offset against one another. The state parameter of the frying medium can thus be determined continuously. Respective event parameters of respective frying events can be used successively in each case to determine a new state parameter of the frying medium. The state of the frying medium can be monitored in this way by means of the determined state parameter.

In one advantageous embodiment, it can be provided that at least one state parameter describing the state of the frying medium is calculated depending on at least one initial value and at least one event parameter describing at least one frying event.

An initial value, for example the state parameter of the frying medium at a specific time, can thus be used and an updated state parameter can be determined on the basis thereof by measuring and processing an event parameter describing at least one frying event. The preceding state is therefore already known and the new state is obtained by measuring and processing an event parameter.

In one advantageous embodiment, it can be provided that the at least one state parameter is and/or correlates with the mass fraction of polar materials in the frying medium. Alternatively or additionally, it can be provided that the at least one state parameter is and/or correlates with the mass fraction of fatty acids in the frying medium.

The aforementioned quality-related parameters, which are already established in practice and form part of quality assurance rules, can thus be determined as such. The measurement of these parameters is therefore superfluous.

In one advantageous embodiment, it can be provided that at least one further state parameter is determined and used to calculate the state parameter. It can additionally be provided that at least one event parameter is used here for the calculation.

The mass fraction of polar materials, for example, can thus be measured and/or determined using a method according to the invention, and can be mathematically combined with at least one, in particular at least two, event parameters in order to determine a more accurate mass fraction of free fatty acids. The same applies conversely to the measurement of the mass fraction of free fatty acids with subsequent determination of the mass fraction of polar materials.

In one advantageous embodiment, it can be provided that the at least one event parameter is the number and/or duration and/or intensity of frying events and/or correlates with at least one of these variables.

The invention has recognized that the quality state of a frying medium is determined not only by any thermal load as a result of maintaining the heat of the frying medium, but above all by thermal and/or chemical reactions occurring during the frying events. A deterioration in the quality of a frying medium results above all from the frying process itself. It therefore suffices to determine the number and/or duration and/or intensity of frying events in order to allow inferences to be made regarding the state parameter and therefore the quality state of a frying food item.

In one advantageous design, it can be provided that the at least one event parameter describes and/or is and/or correlates with a frying food item. It can additionally be provided that the at least one event parameter describes and/or is and/or correlates with the type and/or quantity and/or number of frying food items.

Characteristics of the frying food item can thus be used to determine the state parameter. It is possible, for example, to infer the state parameter from measured characteristics of the frying food item, for example the number of determined frying food items. The aforementioned variables can also be used to simplify the determination of the state parameter.

In one advantageous design, it can be provided that the at least one event parameter is and/or correlates with the quantity of water that has been entrained into the frying medium.

The quantity of entrained water in particular has an effect on the state of the frying food item and is therefore advantageously used to determine the state parameter. The quantity of water can be determined, for example, from the type, quantity and/or number of frying food items.

In one advantageous embodiment, it can be provided that at least one event parameter is calculated and/or corrected using at least one other event parameter.

One event parameter can be, for example, the type of frying food item, and another event parameter can be the number of frying food items. A quantity of entrained water, for example, can be calculated therefrom, from which the state of the frying medium can be derived.

In one advantageous embodiment, it can be provided that a frying event is detected by means of a sensor. It can be provided here that the sensor is a camera.

The frying medium can thus be monitored by means of a camera and a frying event can be read or measured, for example, from a bubble formation or foam formation and can be used to determine the state parameter. The intensity and/or duration of a foam formation, for example, can be used as an event parameter to determine a state parameter of the frying medium.

In one advantageous embodiment, it can be provided that the sensor is an ultrasound sensor. Alternatively or additionally, it can be provided that the sensor is a radar sensor. Alternatively or additionally, it can be provided that the sensor is a lidar sensor.

Frying events can thus be measured in a wide variety of ways, for example on the basis of the foam formation in or on the frying medium, and can be further used to determine the state parameter.

In one advantageous embodiment, it can be provided that at least one event parameter is read from a, preferably digital, point of sale system. Alternatively or additionally, it can be provided that at least one event parameter is calculated by the point of sale system.

The type, quantity and/or number of frying food items can therefore be inferred from orders recorded by the point of sale system, and can be incorporated as event parameters into the determination of the state parameter. A monitoring of frying events by means of, for example, the aforementioned sensors can therefore be superfluous, and the state parameter can be calculated from the monitored frying food items.

In one advantageous embodiment, it can be provided that an insertion of a frying basket is recorded as at least one event parameter.

This can involve the insertion of the frying basket into the fryer. It is thus possible to determine when a frying event begins and when it ends. The number and/or duration of frying events can thus be recorded and used to calculate the state parameter.

Alternatively or additionally, the features focusing on a method for determining the quantity of water entrained into a frying medium during a frying process and/or frying event are provided according to the invention in order to achieve the aforementioned object. In particular, it is therefore proposed according to the invention that a foam formation is measured and evaluated during the frying process and/or frying event in order to achieve the aforementioned object in methods of the type described above.

The foam formation or bubble formation in or on the frying medium can therefore be measured, for example as described above, and can be used to determine the state parameter. The recognition that the state of the frying medium depends mainly on the number, intensity and duration of frying events, and less on the time during which the frying medium is kept at increased temperatures, is reflected herein.

Alternatively or additionally, the features focusing on a detection device for detecting frying events of a fryer having a sensor with a computer preferably fitted over the fryer and/or directed toward the fryer are provided according to the invention in order to achieve the aforementioned objective. In particular, in order to achieve the aforementioned object in detection devices of the type described above, it is therefore proposed according to the invention that the detection device is configured to carry out a method according to the invention.

The aforementioned advantages according to the invention can therefore be used in detection devices.

In one advantageous embodiment, it can be provided that the sensor is a camera and/or an ultrasound sensor and/or a radar sensor and/or a lidar sensor.

The aforementioned sensors can therefore be used to detect frying events. The detection device is thus configured to carry out a method according to the invention.

Alternatively or additionally, the features focusing on a fryer are provided according to the invention in order to achieve the aforementioned object. In particular, in order to achieve the aforementioned object in the case of fryers of the type described above, it is proposed according to the invention that said fryers have a detection device according to the invention.

The advantages of the detection devices according to the invention and methods according to the invention can therefore be used for and with fryers.

### BRIEF DESCRIPTION OF THE DRAWINGS

The invention will now be described in detail with reference to an exemplary embodiment, but is not restricted to the exemplary embodiment. Further exemplary embodiments can be derived from a combination of one or more of the features disclosed in the description and claims with one another and/or with individual features or a plurality of features of the exemplary embodiment.

In the drawings:

FIG. 1 shows a schematic view of the variables influencing the mass fraction of free fatty acids of a frying medium as an example of a quality-related parameter,

FIG. 2 shows the development over time of the mass fraction of free fatty acids of a frying medium depending on frying events that occur, and

FIG. 3 shows a schematic view of a detection device and fryer according to the invention.

### DETAILED DESCRIPTION

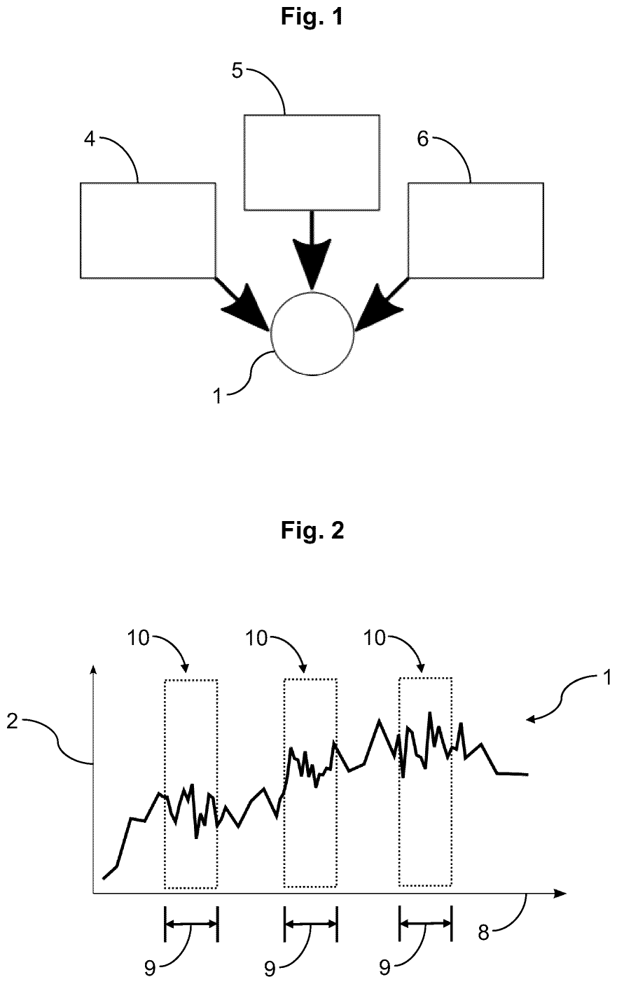

FIG. 1 shows schematically that an increase 1 in the mass fraction of free fatty acids 2 in a frying medium 3 depends mainly on three influencing variables. These variables are hydrolysis 4, migration 5 and polymerization or cracking 6 of free fatty acids. The hydrolysis 4 of fatty acid esters takes place here due to water which is present in the frying food item 7, or is entrained by the frying food item 7 into the frying medium 3. An increase 1 in the mass fraction of free fatty acids 2 further occurs due to migration 4 of free fatty acids from the frying food item 7 into the frying medium 3.

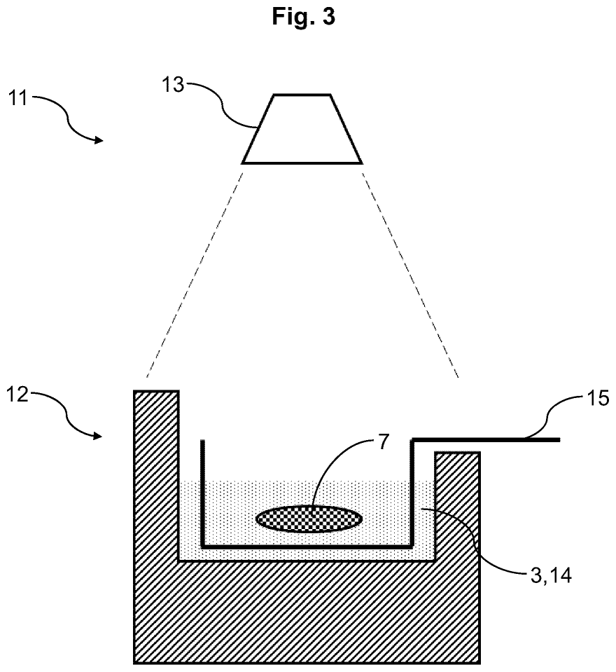

FIG. 2 shows a graphical representation of an example of an increase 1 in the mass fraction of free fatty acids 2 in a frying medium 3 over time 8. It is recognizable that the mass fraction of free fatty acids 2 in a frying medium 3 is determined mainly by the number and duration 9 of frying events 10 that occur and increases over time 8. The mass fraction of free fatty acids 2 does not increase significantly between the frying events 10, wherein the frying medium 3 is merely kept at increased temperatures.

FIG. 3 shows a detection device 11 according to the invention for detecting frying events 10 of a fryer 12 which is a fryer 12 according to the invention here, having a sensor 13 fitted above the fryer 12 and directed toward the fryer 12 and having a computer (not shown in detail here), wherein the detection device 11 is configured to carry out a method according to the invention.

The sensor 13 of the detection device 11 can be a camera and/or an ultrasound sensor and/or a radar sensor and/or a lidar sensor here. The fryer 12 contains a frying oil 14 as an example of a frying medium 3, in which a frying basket 15 is present with the frying food item 7. The methods according to the invention for determining a state parameter of a frying medium 3 can be carried out by means of the detection device 11 and the fryer 12, at least one event parameter which describes at least one frying event 10 is measured and processed for the determination.

It is possible for measured event parameters to be accumulated here. At least one state parameter describing the state of the frying medium 3 can also be calculated depending on at least one initial value and at least one event parameter describing at least one frying event 10. The at least one state parameter can be or can correlate with the mass fraction of polar materials and/or the mass fraction of free fatty acids 2 in the frying medium 3. At least one further state parameter can furthermore be determined and can be used to calculate the state parameter, wherein at least one event parameter can be used for the calculation.

The at least one event parameter can be the number and/or duration 9 and/or intensity of frying events 10, or can correlate with at least one of these variables. The at least one event parameter can describe and/or be a frying food item 7, in particular its type and/or quantity and/or number, and/or can correlate with at least one of these variables. The at least one event parameter can be and/or can correlate with the quantity of water that has been entrained into the frying medium 3. The at least one event parameter can be calculated and/or corrected using at least one other event parameter.

A frying event 10 can be detected here by means of a sensor 13 and preferably by means of a camera. The sensor 13 can also be an ultrasound sensor and/or a radar sensor and/or a lidar sensor. The at least one event parameter can also be read from or calculated by a, preferably digital, point of sale system. An insertion of a frying basket 15, in particular into the fryer 12, can be recorded as the at least one event parameter. The method according to the invention for determining the quantity of water entrained into a frying medium 3 during a frying process and/or frying event 10 can further be carried out with the detection device 11 and the fryer 12 that are shown, wherein a foam formation is measured and evaluated during the frying process and/or frying event 10.

A method for determining a state parameter of a frying medium 3 is therefore proposed, wherein at least one event parameter which describes at least one frying event 10 is measured and processed for the determination. A method for determining the quantity of water entrained into a frying medium 3 during a frying process and/or frying event 10 is further proposed, wherein a foam formation is measured and evaluated during the frying process and/or frying event 10. A detection device 11 for detecting frying events 10 of a fryer 12 having a sensor 13 preferably fitted above the fryer 12 and/or directed toward the fryer 12 and having a computer is further proposed, wherein the detection device 11 is configured to carry out a method according to the invention. A fryer 12 is further proposed which has a detection device 11 according to the invention.

### REFERENCE SIGN LIST

1. Increase
2. Mass fraction of free fatty acids
3. Frying medium
4. Hydrolysis
5. Migration
6. Polymerization or cracking
7. Frying food item
8. Time
9. Duration
10. Frying event
11. Detection device
12. Fryer
13. Sensor
14. Frying oil
15. Frying basket

1. A method for determining at least one state parameter of a frying medium (3), the method comprising:
measuring at least one event parameter which describes at least one frying event (10); and
processing the at least one event parameter that is measured for making a determination the at least one state parameter.

2. The method as claimed in claim 1, further comprising accumulating measured ones of the at least one event parameter.

3. The method as claimed in claim 1, further comprising calculating the at least one state parameter describing the state of the frying medium (3) depending on at least one initial value and the at least one event parameter, wherein the at least one event parameter describes at least one frying event (10).

4. The method as claimed in claim 3, wherein the at least one state parameter is or correlates with at least one of a mass fraction of polar materials or a mass fraction of free fatty acids (2) in the frying medium (3).

5. The method as claimed in claim 3, further comprising determining at least one further state parameter and using the at least one further state parameter for calculating the at least one state parameter.

6. The method as claimed in claim 5, wherein the at least one further parameter is at least one event parameter that is used for the calculation.

7. The method as claimed in claim 1, wherein the at least one event parameter is at least one of a number, a duration, or an intensity of frying events (10) and/or correlates with at least one of the number, the duration, or the intensity of the frying events (10).

8. The method as claimed in claim 1, wherein the at least one event parameter describes and/or is a frying food item (7).

9. The method as claimed in claim 8, wherein the at least one event parameter is at least one of a type, quantity, or number of the frying food item, and/or correlates with at least one of the type, the quantity, or the number of the frying food item (7).

10. The method as claimed in claim 1, wherein the at least one event parameter is and/or correlates with a quantity of water that has been entrained into the frying medium (3).

11. The method as claimed in claim 1, wherein the at least one event parameter is calculated and/or corrected using at least one other event parameter.

12. The method as claimed in claim 1, wherein the frying event (10) is detected by a sensor (13).

13. The method as claimed in claim 12, wherein the sensor (13) is at least one of an ultrasound sensor, a radar sensor, or a lidar sensor.

14. The method as claimed in claim 1, wherein the at least one event parameter is read from and/or calculated by a point of sale system.

15. The method as claimed in claim 1, further comprising recording an insertion of a frying basket (15) as the at least one event parameter.

16. A method for determining the quantity of water entrained into a frying medium (3) during a frying process and/or frying event (10), the method comprising:
measuring and evaluating a foam formation during the frying process and/or frying event (10).

17. A detection device (11) for detecting frying events (10) of a fryer (12), the detection device comprising:
a sensor (13) that is at least one of fitted above the fryer (12) or directed toward the fryer (12), and
a computer configured to carry out the method as claimed in claim 1.

18. The detection device (11) as claimed in claim 17, wherein the sensor (11) is at least one of a camera, an ultrasound sensor, a radar sensor, or a lidar sensor.

19. A fryer (12), comprising the detection device (11) as claimed in claim 17.
